SEQUENCE LISTING (12 sequences)

SEQ ID NO: 1 - DNA, 22 bp, Homo sapiens
  misc (1)..(22): mature micro RNA hsa-miR-320a
aaaagcuggguugagagggcga

SEQ ID NO: 2 - DNA, 24 bp, Homo sapiens
  misc (1)..(24): Part of the precursor micro RNA hsa-mir-320a
gccuucucuucccgguucuucccg

SEQ ID NO: 3 - DNA, 26 bp, Homo sapiens
  misc (1)..(26): Part of the precursor micro RNA hsa-mir-320a
cgggaaaagcuggguugagagggcga

SEQ ID NO: 4 - DNA, 54 bp, Homo sapiens
  misc (1)..(54): Part of the precursor micro RNA mir-320a
gccuucucuucccgguucuucccggagucgggaaaagcuggguugagagggcga

SEQ ID NO: 5 - DNA, 82 bp, Homo sapiens
  misc (1)..(82): hsa-mir-320a
gcuucgcuccccuccgccuucucuucccgguucuucccggagucgggaaaagcuggguugagagggcgaaaaaggaugaggu

SEQ ID NO: 6 - DNA, 81 bp, Pan troglodytes
  misc (1)..(81): ptr-mir-320a
gcuucgcuccucuccgccuucucuucccgguucuucccggagucgggaaaagcuggguugagagggcgaaaaaggaugagg

SEQ ID NO: 7 - DNA, 82 bp, Pongo pygmaeus; identical to SEQ ID NO: 5
  misc (1)..(82): ppy-mir-320a

SEQ ID NO: 8 - DNA, 82 bp, Bos taurus
  misc (1)..(82): bta-mir-320
aaaaacgaaaaagaggccuucucuucccgguucuucccggagucgggaaaagcuggguugagagggcgaaaaaggaagaggg

SEQ ID NO: 9 - DNA, 54 bp, Canis familiaris; identical to SEQ ID NO: 4
  misc (1)..(54): cfa-mir-320

SEQ ID NO: 10 - DNA, 82 bp, Mus musculus
  misc (1)..(82): mmu-mir-320
gccucgccgcccuccgccuucucuucccgguucuucccggagucgggaaaagcuggguugagagggcgaaaaaggauguggg

SEQ ID NO: 11 - DNA, 82 bp, Rattus norvegicus
  misc (1)..(82): rno-mir-320
gccucgcuguccuccgccuucucuucccgguucuucccggagucgggaaaagcuggguugagagggcgaaaaaggauauggg

SEQ ID NO: 12 - DNA, 81 bp, Macaca mulatta
  misc (1)..(81): mml-mir-320
gcuucgcuccccuccgccuucucuucccgguucuucccggagucgggaaaagcuggguugagagggcgaaaaaggaugagg

(19) United States
(12) Patent Application Publication
(10) Pub. No.: US 2014/0199402 A1
(43) Pub. Date: Jul. 17, 2014

(54) NOVEL COMPOUNDS FOR THE TREATMENT OF INFLAMMATORY BOWEL DISEASE

(71) Applicants: Alexander Schmidt, Havixbeck (DE); Katharina Veltman, Muenster (DE); Christoph Cichon, Everswinkel (DE)

(72) Inventors: Alexander Schmidt, Havixbeck (DE); Katharina Veltman, Muenster (DE); Christoph Cichon, Everswinkel (DE)

(21) Appl. No.: 14/238,443

(22) PCT Filed: Aug. 9, 2012
(86) PCT No.: PCT/EP2012/065568

§ 371 Date: Feb. 11, 2014

(30) Foreign Application Priority Data
Aug. 12, 2011 (EP) 11006650.3
Oct. 18, 2011 (EP) 11185604.3

(51) Int. Cl. C12N 15/113; A23L 1/30; A61K 35/74

(52) U.S. Cl. CPC C12N 15/113; A61K 35/741; A23L 1/3012
USPC 424/490; 514/44 A; 424/93.2

(57) ABSTRACT

The present invention relates to a nucleic acid molecule of up to 150 nucleotides comprising consecutively from 5′ to 3′ (a) a first part whose sequence is between 50% and 100% complementary to the sequence AAAAGCUGGGUUGAGAGGGCGA; (b) a second part capable of forming a loop between the first and the third part; and (c) a third part comprising or consisting of the sequence AAAAGCUGGGUUGAGAGGGCGA; for use as a medicament. The present invention further relates to a nucleic acid molecule of up to 25 nucleotides comprising the sequence AAAAGCUGGGUUGAGAGGGCGA, for use as a medicament. In another aspect, the present invention relates to a composition comprising at least one mature miRNA selected from the group consisting of hsa-miR-320a, ptr-miR-320a, ppy-miR-320a, bta-miR-320, cfa-miR-320, mmu-miR-320, rno-miR-320, and mml-miR-320, and/or one or more mir-RNA precursor(s) thereof, for use as a medicament.

The present invention relates to a (preferably isolated) nucleic acid molecule of up to 150 nucleotides comprising consecutively from 5′ to 3′ (a) a first part whose sequence is between 50% and 100% complementary to the sequence AAAAGCUGGGUUGAGAGGGCGA; (b) a second part capable of forming a loop between the first and the third part; and (c) a third part comprising or consisting of the sequence AAAAGCUGGGUUGAGAGGGCGA; for use as a medicament. The present invention further relates to a nucleic acid molecule of up to 25 nucleotides comprising the sequence AAAAGCUGGGUUGAGAGGGCGA, for use as a medicament. In another aspect, the present invention relates to a composition comprising at least one mature miRNA selected from the group consisting of hsa-miR-320a, ptr-miR-320a, ppy-miR-320a, bta-miR-320, cfa-miR-320, mmu-miR-320, rno-miR-320, and mml-miR-320, and/or one or more mir-RNA precursor(s) thereof, for use as a medicament.

The intestinal mucosa is the first epithelial layer of the gastrointestinal tract on the luminal side. This layer comes in direct contact with microorganisms residing in the intestine and therefore constitutes the largest and most important barrier against the external environment. It acts as a selectively permeable barrier, permitting the absorption of nutrients, electrolytes, and water while maintaining an effective defense against intraluminal toxins, antigens, and enteric flora.

The epithelium maintains its selective barrier function through the formation of complex protein-protein networks that mechanically link adjacent cells and seal the intercellular space, the so called tight junctions. The tight junction, also called zona occludens, is a specialized cell-cell interaction that is found in almost all types of epithelial cells in different organs in the body. Tight junctions are the closely associated areas of two adjacent cells whose membranes join together forming a virtually impermeable barrier to gastrointestinal contents. A tight junction comprises densely packed protein complexes that provide contact between the membranes of two adjacent cells. One of the functions of tight junctions is regulating the passage of molecules and ions through the space between cells. The tight junction also represents a major barrier for paracellular transport, i.e. transport through the intercellular spaces between epithelial cells, and may prevent such passage of molecules and ions. Consequently, materials must enter the epithelial cells, through e.g. diffusion or active transport, in order to pass through the tissue. This is called transcellular transport and such transport provides control over what substances are allowed through e.g. the intestinal mucosa. Epithelia are classed as ‘tight’ or ‘leaky’ depending on the ability of the tight junctions to prevent water and solute movement through intercellular space.

An important task of the intestine is to form a defensive barrier to prevent absorption of damaging substances from the external environment. This protective function is mainly dependent on the barrier properties of the intestinal mucosa. The permeability of the intestinal mucosa is determined at least in part by the strength of the tight junctions of the intestinal epithelial cells.

There are a number of factors that may affect tight junctions, including food components such as gluten and casein in some individuals. However, also infectious organisms such as specific pathogenic strains of E. coli, Salmonella and C. difficile have the ability to disrupt the tight junction protein complexes between the epithelial cells and setting up an infection. Disruption of the tight junctions may result in lowering the barrier properties of the intestinal mucosal epithelium, leading to leaky gut.

Dysfunction of the gut barrier of intestinal mucosa, as encountered by animals, including fish, due to disruption of tight junctions in stressful situations and/or during immuno-suppression may result in septicemia, and/or toxemia, leading to a decreased feed efficiency in animals or food uptake in humans.

Currently little or no attention is paid in animal nutrition to the gut barrier properties. Treatments or nutritional supplementations to improve the mucosal integrity are largely unknown. However, there have been sporadic reports suggesting that specific nutrients such as the amino acid glutamine may help in a decreasing in gut permeability and may lead to an improved functioning of the mucosal barrier.

The technical problem underlying the present invention is to provide means and methods which help to enhance the transepithelial electrical resistance of the intestinal mucosa.

The present invention addresses this need and thus provides, as a solution to the technical problem, an, preferably isolated, nucleic acid molecule which either consists of or comprises the sequence AAAAGCUGGGUUGAGAGGGCGA (from 5′to 3′) for use as a medicament. The term “medicament” as used herein is equivalent to the term “pharmaceutical composition”.

Further embodiments of the present invention are characterized and described herein and also reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein. Unless otherwise indicated, the term “at least” preceding a series of elements is to be understood to refer to every element in the series. At least one includes for example, one, two, three, four, or five or even more.

Those skilled in the art will recognize, or be able to ascertain using no more than routine experimentation, many equivalents to the specific embodiments of the invention described herein. Such equivalents are intended to be encompassed by the present invention. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Provided that the present specification refers to a defined nucleic acid sequence, said sequence is depicted in its 5′ to 3′orientation (unless otherwise specified in the text).

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

MicroRNAs (miRNAs) are small, RNA molecules encoded in the genomes of plants and animals. These highly conserved, 21-27-mer RNAs regulate the expression of genes by binding to the 3′-untranslated regions (3′-UTR) of specific mRNAs. Several research groups have provided evidence that miRNAs may act as key regulators of processes as diverse as early development, cell proliferation and cell death, apoptosis and fat metabolism, and cell differentiation. There is speculation that in higher eukaryotes, the role of miRNAs in regulating gene expression could be as important as that of transcription factors. The role of miRNA in the regulation of tight junction proteins has, up to the present specification, not been investigated.

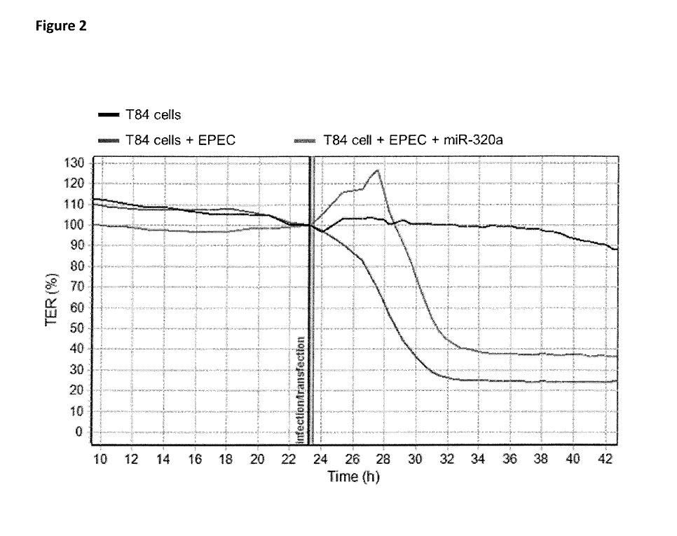

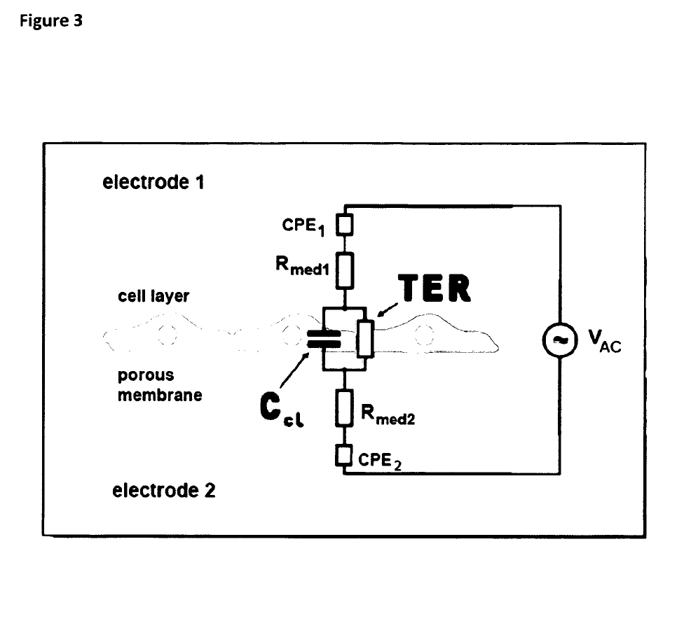

It has been found by the present inventors that it is possible to positively influence the transepithelial electrical resistance (TER) of epithelial cells with miRNA 320a. Using model cellular barriers (polarized T84 cells—ATCC No. CCL-248) it has been demonstrated by the present inventors that this miRNA is able to prevent the barrier-disrupting effect of the enteropathogenic E. coli (EPEC) prototype strain E2348/69 and is furthermore able to restore the integrity of the epithelial barrier after disruption by EPEC E2348/69. This can be illustrated by observing the transepithelial electrical resistance which represents a parameter of barrier integrity (see FIGS. 2 and 3 for further illustration).

The negative effect on the integrity of the epithelial barrier exerted by the EPEC strain E2348/69 could be abrogated by employing the miRNA described in this application by transfecting T84 cells with the according miRNA after co-incubation with EPEC bacteria (see FIG. 2).

Thus, in a first aspect, the present invention relates to a medicament comprising a nucleic acid molecule consisting of or comprising the sequence AAAAGCUGGGUUGAGAGGGCGA (from 5′to 3′).

The sequence AAAAGCUGGGUUGAGAGGGCGA is equivalent to a mature microRNA (miRNA) which can be found in the respective databases (for example www.mirbase.org) under the following non-limiting denominations: hsa-miR-320a (Accession number MIMAT0000510), ptr-miR-320a, ppy-miR-320a, bta-miR-320, cfa-miR-320, mmu-miR-320, rno-miR-320, and/or mml-miR-320. The species of origin is thereby designated with a three-letter prefix, e.g., hsa-miR-320a would be from human (Homo sapiens) and mmu-miR-320a would be a mouse (Mus musculs) miRNA. Other mature miRNAs might come up in the future and all these miRNAs are also within the scope of the present invention, provided that they consist of the sequence AAAAGCUGGGUUGAGAGGGCGA. It follows that the sequence AAAAGCUGGGUUGAGAGGGCGA as used herein can be replaced with any miRNA sequence selected from the group consisting of hsa-miR-320a, ptr-miR-320a, ppy-miR-320a, bta-miR-320, cfa-miR-320, mmu-miR-320, rno-miR-320, and/or mml-miR-320 (or future miRNAs from other species or from different places in the genome).

It will be understood, however, that irrespective of the nomenclature of the miRNAs, the present invention encompasses all nucleic acid sequences which consist of the isolated sequence AAAAGCUGGGUUGAGAGGGCGA (either synthetically manufactured or naturally processed) and any precursor of said sequence, provided that the precursor leads to the expression or provision of the isolated sequence AAAAGCUGGGUUGAGAGGGCGA intracellularily, preferably in a eucaryotic cell, more preferably in a mammalian cell and most preferred in a human cell. miRNA genes are usually transcribed by RNA polymerase II. The product, which is called primary miRNA (pri-miRNA), may be hundreds or thousands of nucleotides in length and typically contains one or more miRNA stem loops. It is presently accepted that Pasha, also known as DGCR8 is required for microRNA processing. It binds to Drosha, an RNase III enzyme, to form a Microprocessor complex that cleaves the pri-miRNA to the characteristic stem-loop structure of the pre-miRNA, which is then further processed to miRNA fragments by the enzyme Dicer and subsequently incorporated into the RNA-induced silencing complex (RISC). The pre-miRNA is frequently characterized by a two-nucleotide overhang at its 3′ end and 3′ hydroxyl and 5′ phosphate groups.

The “precursors” of the present invention thus include pri-miRNAs and pre-miRNAs which upon processing in a cell (preferably a mammalian cell and more preferably in a human cell) lead to the mature miRNA nucleic acid sequence AAAAGCUGGGUUGAGAGGGCGA.

However, also artificial precursors are within the scope of the present invention, provided that these artificial precursors are processable within a cell (preferably a mammalian cell and more preferably in a human cell) to the nucleic acid sequence AAAAGCUGGGUUGAGAGGGCGA. Means and methods to test whether a given precursor is processable to the sequence AAAAGCUGGGUUGAGAGGGCGA are within the means and expertise of the skilled person. To this end it is for example possible to specifically capture the processed target sequence AAAAGCUGGGUUGAGAGGGCGA and/or to amplify the respective sequence by means of standard PCR-amplification techniques, and thereby to evaluate whether a precursor is indeed processable to said target sequence or not. Commercially available assays may be used in this regard, which assays are meanwhile offered by many companies including QIAGEN.

“Processable precursors” or “precursors which are processable” etc., as disclosed herein, thus includes natural and/or artificial (synthetic) precursor molecules which are processed intracellularily by either all or a selection of the respective miRNA processing steps, and which result in the desired miRNA (equivalent to the sequence AAAAGCUGGGUUGAGAGGGCGA)—these non-limiting miRNA processing steps may include inter alia: transcription of miRNA genes by RNA polymerase II; processing by Pasha/DGCR8 and Drosha, an RNase III enzyme, to form a Microprocessor complex that cleaves the pri-miRNA to the characteristic stem-loop structure of the pre-miRNA, which is then further processed to miRNA fragments by the enzyme Dicer and subsequently incorporated into the RNA-induced silencing complex (RISC). The miScript miRNA Mimics provided by QIAGEN, for example, need no processing by Pasha, Drosha and/or Dicer but simply interact with the RISC complex and, thereby, become a functional mature miRNA. In other words, these artificial precursors merely need the step of integration into the RISC complex, i.e. said precursor is “processable” because a cell is able to process these artificial precursors into a mature miRNA.

A processable precursor is preferably characterized by one or more of the following structural and functional characteristics:
(a) the precursor is capable of forming a stem-loop (a double helix that ends in an unpaired loop—it occurs when two regions of the same strand, usually at least in part complementary in nucleotide sequence when read in opposite directions, base-pair to form a double helix that ends in an unpaired loop);
(b) the precursor is processable (cleavable) by Dicer;
(c) the precursor is at least in part double stranded;
(d) the precursor contains a part (third part) which is identical to the mature miRNA (equivalent to the sequence AAAAGCUGGGUUGAGAGGGCGA) and a further part (first part) which is at least partially complementary thereto;
(e) the third part and the first part (see (d)) are spaced apart by a second part;
(f) at least the first and the third part of the precursor (see (d)) are made out of nucleotides;
(g) some or all of said nucleotides mentioned in (f) can be modified (such modifications include for example those that are detailed in WO 2006/137941, preferably those mentioned on pages 48 and 49—the term “modification” is also explained in more detail herein elsewhere);
(h) the precursor can be cleaved by Drosha; and/or
(i) the precursor can be transported across the nucleolemma by a karyopherin, preferably by Exportin-5.

Precursors which are characterized by at least the above mentioned characteristic (c) or (d) are preferred. Precursors which are characterized by at least the above mentioned characteristic (d) and (c) are more preferred. Precursors which are characterized by at least the above mentioned characteristic (d) and (c) and (f) are even more preferred.

Artificial precursor molecules which can be processed to the desired sequence AAAAGCUGGGUUGAGAGGGCGA are also envisaged, for example artificial precursors which are meanwhile offered and constructed by QIAGEN (miScript miRNA Mimics) or other well-known companies. All these artificial precursors are processable intracellularily and lead to the isolated sequence AAAAGCUGGGUUGAGAGGGCGA which is equivalent to the mature miRNA hsa-miRNA-320a or the other mature miRNAs mentioned herein.

“Processable” thus means in essence that all the precursors mentioned herein can be processed intracellularily to the isolated sequence AAAAGCUGGGUUGAGAGGGCGA. As mentioned before, said nucleic acid molecule is preferably processable by a mammalian cell and most preferred by a human cell.

It is envisaged that, within the context of all embodiments of the present invention, one or more or even all of the nucleotide(s) “U” of the sequence AAAAGCUGGGUUGAGAGGGCGA (or any other sequence disclosed herein) can be replaced by the nucleotide “T”.

The medicament of the present invention is preferably used for the treatment and/or amelioration, or prevention of a disease, which disease is characterized by a reduction or loss of the intestinal barrier function as mediated by the intestinal mucosa. The permeability of the intestinal mucosa is determined at least in part by the strength of the tight junctions of the intestinal epithelial cells and the diseases mentioned herein are therefore characterized by a disruption, reduction or loss of the tight junction protein complexes between the epithelial cells of the intestinal mucosa. Disruption, reduction or loss of the tight junctions may inter alia result in intestinal hyperpermeability which is characterized by a reduction or loss of the barrier function of the intestinal mucosal epithelium, leading to a so-called “leaky gut”.

In vivo permeability can conveniently be assessed by measuring the permeation of sugars, such as D-xylose, mannitol, rhamnose or lactulose, across the mucosa and detecting the recovery in the urine. In a number of studies using different markers, like D-xylose, mannitol and lactulose, as part of a sugar absorption/permeability tests, abnormal small intestinal absorption was demonstrated.

The skilled person is thus well aware how to test for a reduction or loss of the intestinal barrier function (see for example BioHealth Diagnostics in San Diego, USA which offers a commercially available test), i.e. the skilled person can easily decide whether a disease is a disease which is characterized by a reduction or loss of the intestinal barrier function, or not.

Preferred diseases which are to be treated, ameliorated or prevented in the context of the present invention (therapeutically or prophylactically) are selected from diseases which can be subsumed under the collective term inflammatory bowel disease (IBD), ulcerative colitis and Crohn's disease being particularly preferred.

The term “inflammatory bowel disease” or “IBD” as used herein is a collective term describing inflammatory disorders of the gastrointestinal tract, the most common forms of which are ulcerative colitis and Crohn's disease. The present invention provides pharmaceutical compositions and methods for treatment of IBD of any etiology. In certain embodiments, the present invention provides methods for treating ulcerative colitis, Crohn's disease, diversion colitis, ischemic colitis, infectious colitis, chemical colitis, microscopic colitis (including collagenous colitis and lymphocytic colitis), atypical colitis, pseudomembranous colitis, fulminant colitis, autistic enterocolitis, indeterminate colitis, Behcet's disease, gastroduodenal CD, jejunoileitis, ileitis, ileocolitis, Crohn's (granulomatous) colitis, irritable bowel syndrome, mucositis, radiation induced enteritis, short bowel syndrome, stomach ulcers, diverticulitis, pouchitis, proctitis, and chronic diarrhea. Reference to IBD throughout the specification is sometimes referred to in the specification as exemplary of gastrointestinal inflammatory conditions, and is not meant to be limiting.

It will be understood that the compounds and compositions of the present invention are for use in the treatment of certain medical conditions (disclosed herein). The present invention also relates to methods of treatment comprising the step of administering the compounds, nucleic acid molecules, vectors and/or host cells of the present invention (either alone or in admixture) to a subject in need thereof, typically to a subject suffering from the diseases mentioned herein. The “subject” typically includes mammals, and in particular human beings, cats, dogs, camels, horses, sheep, cows, apes, pigs, guinea pigs, goats etc., human beings being preferred.

The nucleic acid molecules, vectors, host cells and/or compositions of the present invention may be used in a therapeutic or prophylactic medical setting.

The present invention thus relates in a specific embodiment to a nucleic acid molecule of up to 150 nucleotides comprising consecutively from 5′ to 3′:
(a) a first part whose sequence is between 50% and 100% complementary to the sequence AAAAGCUGGGUUGAGAGGGCGA;
(b) optionally a second part connecting said first and third part; and
(c) a third part comprising the sequence AAAAGCUGGGUUGAGAGGGCGA;
   for use as a medicament, and in particular for use in the treatment and/or amelioration, or prevention of a disease which disease is characterized by a reduction or loss of the intestinal barrier function as mediated by the intestinal mucosa. Said nucleic acid molecule characterizes some precursors of the present invention.

The first part comprises or consists of a nucleic acid sequence which is between 50% and 100% complementary to the sequence AAAAGCUGGGUUGAGAGGGCGA over the entire length of said sequence (i.e. 22 nucleotides)—in a preferred embodiment, said first part consist of or comprises a nucleic acid sequence which is characterized by four, five, sixor seven nucleotides which are not complementary to the sequence AAAAGCUGGGUUGAGAGGGCGA while the remaining 18, 17, 16, or 15 nucleotides are complementary thereto (resulting in an about 68 to about 82% complementary sequence). In a more preferred embodiment, said first part comprises mismatches to the seven highlighted (bold and in italics) parts of sequence AAAAGCUGGGUUGAGAGGGCGA, while the remaining nucleotides are complementary thereto.

In a more preferred embodiment, said first part comprises at least 4,5, 6, 7, 8, 9, 10, 11, 12, 13, 14, 15, 16, 17, 18, 19, 20, 21, 22, 23, or 24 consecutive nucleotides of the sequence GCCUUCUCUUCCCGGUUCUUCCCG (from 5′to 3′) which is in part complementary to the sequence AAAAGCUGGGUUGAGAGGGCGA. It is also envisaged that the last nucleotide of the first part which is adjacent to the second part is a “G”.

In another embodiment, said first part is between 50 to 100% complementary to the third part (over the entire length of said third part).

It is preferred that the first part is about 24 to 75, more preferred about 24 to 50 and even more preferred about 24 to 40 nucleotides in length. A length of 39 nucleotides is particularly preferred, as it resembles the first part in the pre-miRNA precursor hsa-miR-320a (Accession number MI0000542).

The optional second part connects the first and the third part. It will be understood, however, that the connection of the first and the third part is not mandatory—see for example the miScript miRNA Mimics provided by QIAGEN—these constructs have no linker between the first and the third part.

The optional second part is preferably capable of forming a loop between the first and the third part. In a preferred embodiment, said second part is or comprises a nucleic acid sequence which is about 3 to 30 nucleotides in length, four nucleotides in length being preferred. Said nucleotides of the second part are unpaired, thereby forming a loop structure. In a preferred embodiment, said second part nucleotide sequence consist or comprises the nucleotide sequence (5′to 3′) GAGU.

It is also envisaged that the second part is entirely or in part replaced by a chemical linker. Such linkers which are capable of connecting two nucleic acid sequences are well known to the skilled person.

The third part comprises or consists of the sequence AAAAGCUGGGUUGAGAGGGCGA. It is preferred that the third part is about 22 to 75, more preferred about 22 to 50 and even more preferred about 22 to 40 nucleotides in length. A length of 39 nucleotides is particularly preferred, as it resembles the third part in the pre-miRNA precursor hsa-miR-320a (Accession number MI0000542). In a preferred embodiment, said third part further comprises the sequence CGGG upstream of the sequence AAAAGCUGGGUUGAGAGGGCGA, and in a more preferred embodiment directly upstream of the sequence AAAAGCUGGGUUGAGAGGGCGA, i.e. the third part then comprises the sequence CGGGAAAAGCUGGGUUGAGAGGGCGA. It is also envisaged that the “C” in the before mentioned CGGG is the last nucleotide of the third part which is adjacent to the second part.

The term “up to 150 nucleotides” encompasses nucleic acid molecules having a total length of about 150 nucleotides or below, e.g. 145, 140, 135, 130, 125, 120, 115, 110, 105, 100, 99, 98, 97, 96, 95, 94, 93, 92, 91, 90, 89, 88, 87, 86, 85, 84, 83, 82, 81, 80, 79, 78, 77, 76, 75, 70, 65, 60, 55, 50, 45, 40 or even below.

A length of about 90 nucleotides or below is preferred and a length of 82 nucleotides is more preferred as it resembles the length of the pre-miRNA precursor hsa-miR-320a (Accession number MI0000542). In another more preferred embodiment said nucleic acid molecule is up to 54 nucleotides which is the length of cfa-mir-320 (accession number MI0008063). It is therefore also envisaged that the nucleic acid molecule of the present invention is between 54 and 82 nucleotides in length.

In a further embodiment, said nucleic molecule of up to 150 nucleotides is a precursor of hsa-miR-320a, ptr-miR-320a, ppy-miR-320a, bta-miR-320, cfa-miR-320, mmu-miR-320, rno-miR-320, and/or mml-miR-320. It is well known that miRNAs are derived from the endogenously produced pre-miRNA (precursor) of about 75-90 nucleotides in length having a hairpin or stem-loop structure as explained herein elsewhere. The present invention thus includes all endogenously produced precursors of the miRNA sequence AAAAGCUGGGUUGAGAGGGCGA. The precursor hsa-mir-320a, ptr-mir-320a, ppy-mir-320a, bta-mir-320, cfa-mir-320, mmu-mir-320, rno-mir-320, and/or mml-mir-320 are particularly envisaged. The uncapitalized “mir-” thereby refers to the pre-miRNA, while a capitalized “miR-” refers to the mature form. Thus, in a further embodiment the present invention relates to a composition comprising at least one miRNA selected from the group consisting of hsa-miR-320a, ptr-miR-320a, ppy-miR-320a, bta-miR-320, cfa-miR-320, mmu-miR-320, rno-miR-320, and mml-miR-320, and/or one or more mir-RNA precursor(s) thereof, for use as a medicament, and in particular for use in the treatment and/or amelioration, or prevention of a disease which disease is characterized by a reduction or loss of the intestinal barrier function as mediated by the intestinal mucosa.

In a further embodiment of the nucleic acid molecules of up to 150 nucleotides of the present invention, said nucleic acid molecule comprises the sequence GCCUUCUCUUCCCGGUUCUUCCCGGAGUCGGGAAAAGCUGGGUUGAGAGGGCGA.

In another embodiment, the present invention relates to nucleic acid molecule of up to 150 nucleotides which is characterized by a nucleic acid sequence comprising or consisting of any one of the following sequences:

(hsa-mir-320a)
GCUUCGCUCCCCUCCGCCUUCUCUUCCCGGUUCUUCCCGGAGUCGGGAAA
AGCUGGGUUGAGAGGGCGAAAAAGGAUGAGGU;
(ptr-mir-320a)
GCUUCGCUCCUCUCCGCCUUCUCUUCCCGGUUCUUCCCGGAGUCGGGAAA
AGCUGGGUUGAGAGGGCGAAAAAGGAUGAGG;
(ppy-mir-320a)
GCUUCGCUCCCCUCCGCCUUCUCUUCCCGGUUCUUCCCGGAGUCGGGAAA
AGCUGGGUUGAGAGGGCGAAAAAGGAUGAGGU;
(bta-mir-320)
AAAAACGAAAAAGAGGCCUUCUCUUCCCGGUUCUUCCCGGAGUCGGGAAA
AGCUGGGUUGAGAGGGCGAAAAAGGAAGAGGG;
(cfa-mir-320)
GCCUUCUCUUCCCGGUUCUUCCCGGAGUCGGGAAAAGCUGGGUUGAGAGG
GCGA;
(mmu-mir-320)
GCCUCGCCGCCCUCCGCCUUCUCUUCCCGGUUCUUCCCGGAGUCGGGAAA
AGCUGGGUUGAGAGGGCGAAAAAGGAUGUGGG;
(rno-mir-320)
GCCUCGCUGUCCUCCGCCUUCUCUUCCCGGUUCUUCCCGGAGUCGGGAAA
AGCUGGGUUGAGAGGGCGAAAAAGGAUAUGGG; and
(mml-mir-320)
GCUUCGCUCCCCUCCGCCUUCUCUUCCCGGUUCUUCCCGGAGUCGGGAAA
AGCUGGGUUGAGAGGGCGAAAAAGGAUGAGG,

for use as a medicament, and in particular for use in the treatment and/or amelioration, or prevention of a disease which disease is characterized by a reduction or loss of the intestinal barrier function as mediated by the intestinal mucosa. It is envisaged that the above sequences comprise up to 10 nucleotide exchanges (substitutions, deletions, insertions, substitutions being preferred) in comparison to the above depicted nucleic acid sequences, provided that the exchanges are located outside the nucleotide sequence AAAAGCUGGGUUGAGAGGGCGA. “Up to 10 exchanges” includes 1, 2, 3, 4, 5, 6, 7, 8, 9 or 10 deletions, substitutions or insertions, provided that these exchanges are located outside the nucleotide sequence AAAAGCUGGGUUGAGAGGGCGA, more preferably outside the nucleotide sequence CGGGAAAAGCUGGGUUGAGAGGGCGA, even more preferred outside the nucleotide sequence CGGGAAAAGCUGGGUUGAGAGGGCGA and CCGCCUUCUCUUCCCGGUUCUUCCCG and most preferred outside the nucleotide sequence GCCUUCUCUUCCCGGUUCUUCCCGGAGUCGGGAAAAGCUGGGUUGAGAGGGCGA.

Nucleic acid molecules which are capable of hybridizing under stringent conditions to a sequence which is complementary to hsa-miR-320a, ptr-miR-320a, ppy-mir-320a, bta-miR-320, cfa-miR-320, mmu-miR-320, rno-miR-320, and/or mml-miR-320 are also envisaged. It will be understood that these hybridizing nucleic acid molecules comprise the sequence AAAAGCUGGGUUGAGAGGGCGA. In an even further embodiment these nucleic acid molecule are up to 22, 23, 24, 25, or 26 nucleotides in length and comprise the sequence AAAAGCUGGGUUGAGAGGGCGA. The aforementioned hybridizing nucleic acid molecules are intended for use as a medicament, and in particular for use in the treatment and/or amelioration, or prevention of a disease which disease is characterized by a reduction or loss of the intestinal barrier function as mediated by the intestinal mucosa.

Nucleic acid molecules which are capable of hybridizing under stringent conditions to a sequence which is complementary to hsa-mir-320a ptr-mir-320a, ppy-mir-320a, bta-mir-320, cfa-mir-320, mmu-mir-320, rno-mir-320, and/or mml-mir-320 are also envisaged. It will be understood that these hybridizing nucleic acid molecules comprise the sequence AAAAGCUGGGUUGAGAGGGCGA-. These hybridizing nucleic acid molecules are preferably “processable precursors” (explained herein elsewhere) and may therefore be further characterized by one or more of the following structural and functional characteristics:
(a) the precursor is capable of forming a stem-loop (a double helix that ends in an unpaired loop—it occurs when two regions of the same strand, usually at least in part complementary in nucleotide sequence when read in opposite directions, base-pair to form a double helix that ends in an unpaired loop);
(b) the precursor is processable (cleavable) by Dicer;
(c) the precursor is at least in part double stranded;
(d) the precursor contains a part (third part) which is identical to the mature miRNA (equivalent to the sequence AAAAGCUGGGUUGAGAGGGCGA) and a further part (first part) which is at least partially complementary thereto;
(e) the third part and the first part (see (d)) are spaced apart by a second part;
(f) at least the first and the third part of the precursor (see (d)) are made out of nucleotides;
(g) some or all of said nucleotides mentioned in (f) can be modified (such modifications include for example those that are detailed in WO 2006/137941, preferably those mentioned on pages 48 and 49—the term “modification” is also explained in more detail herein elsewhere);
(h) the precursor can be cleaved by Drosha;
(i) the precursor can be incorporated into the RISC complex.

The above mentioned nucleic acid molecules are in a preferred embodiment capable of hybridizing to hsa-mir-320a under stringent conditions.

Nucleic acid molecules which are characterized by at least the above mentioned characteristic (d) are preferred. Nucleic acid molecules which are characterized by at least the above mentioned characteristic (d) and (c) are more preferred. Nucleic acid molecules which are characterized by at least the above mentioned characteristic (d) and (c) and (f) are even more preferred. All the aforementioned hybridizing nucleic acid molecules are intended for use as a medicament, and in particular for use in the treatment and/or amelioration, or prevention of a disease which disease is characterized by a reduction or loss of the intestinal barrier function as mediated by the intestinal mucosa.

As used herein, the term “hybridizes under stringent conditions” is intended to describe conditions for hybridization and washing under which nucleotide sequences at least 50% homologous to each other typically remain hybridized to each other. The conditions can be such that sequences at least about 65%, at least about 70%, or at least about 75% or at least about 85% or at least about 95% or more homologous to each other typically remain hybridized to each other. Such stringent conditions are known to those skilled in the art and can be found in Current Protocols in Molecular Biology, John Wiley & Sons, N. Y. (1989), 6. 3.1-6.3.6. One example of stringent hybridization conditions are hybridization in 6X sodium chloride/sodium citrate (SSC) at about 45° C., followed by one or more washes in 0.2×SSC, 0.1% SDS at 50-65° C.

In further embodiments, said nucleic acid molecules which are capable of hybridizing under stringent conditions to hsa-miR-320a (which is preferred), hsa-mir-320a (which is likewise preferred), ptr-miR-320a, ptr-mir-320a, ppy-miR-320a, ppy-mir-320a, bta-miR-320, bta-nnir-320, cfa-miR-320, cfa-mir-320, mmu-miR-320, mmu-mir-320, rno-miR-320, rno-mir-320, and/or mml-miR-320 can be further characterized as follows:
(i) they comprise the sequence GAGU upstream (towards the 5′end) of the sequence AAAAGCUGGGUUGAGAGGGCGA; and/or
(ii) they comprise the sequence CGGG upstream (towards the 5′end) of the sequence AAAAGCUGGGUUGAGAGGGCGA; and/or
(iii) they comprise from 5′ to 3′ the sequences GAGU, CGGG and AAAAGCUGGGUUGAGAGGGCGA; and/or
(iv) they comprise the sequence GCCUUCUCUUCCCGGUUCUUCCCG (upstream of AAAAGCUGGGUUGAGAGGGCGA); and/or
(v) they comprise the sequence GCCUUCUCUUCCCGGUUCUUCCCGGAGUCGGGAAAAGCUGGGUUGAGAGGGCGA.

In a further embodiment, said hybridizing nucleic acid molecules are up to 150 nucleotides in length.

In an even further embodiment, the present invention relates nucleic acid molecule of up to 22, 23, 24, 25, or 26 nucleotides in length and comprising the sequence AAAAGCUGGGUUGAGAGGGCGA, for use as a medicament, and in particular for use in the treatment and/or amelioration, or prevention of a disease which disease is characterized by a reduction or loss of the intestinal barrier function as mediated by the intestinal mucosa.

The present invention also relates to a vector comprising the nucleic acid molecules, sequences, precursors, or fragments of the invention (in particular a nucleic acid molecule consisting of or comprising the sequence AAAAGCUGGGUUGAGAGGGCGA), for use as a medicament, and in particular for use in the treatment and/or amelioration, or prevention of a disease which disease is characterized by a reduction or loss of the intestinal barrier function as mediated by the intestinal mucosa.

“Vector” as used herein refers to a recombinant DNA or RNA plasmid or virus that comprises a heterologous nucleic acid sequence capable of being delivered to a target cell, either in vitro, in vivo or ex-vivo. The nucleic acid sequence can be operably linked to another nucleic acid sequence such as promoter or enhancer and may control the transcription of the nucleic acid sequence of interest. As used herein, a vector need not be capable of replication in the ultimate target cell or subject. The term vector may include expression vector and cloning vector. An “expression vector” refers to a recombinant DNA or RNA construct, such as a plasmid, a phage, recombinant virus or other vector that, upon introduction into an appropriate host cell, results in expression of the inserted DNA. Appropriate expression vectors include those that are replicable in eukaryotic cells and/or prokaryotic cells and those that remain episomal or those which integrate into the host cell genome.

The term “vector” or “expression vector” is used herein thus means nucleic acid based vectors which are used in accordance with the present invention as a vehicle for introducing into and expressing a the nucleic acids molecules of the instant invention (in particular a nucleic acid molecule consisting of or comprising the sequence AAAAGCUGGGUUGAGAGGGCGA) in a host cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene, and the ability to enter and/or replicate in eukaryotic or prokaryotic cells. Additionally elements may also be included in the vector such as signal sequences, splice signals, as well as transcriptional promoters, enhancers, and termination signals. Examples of suitable vectors include, but are not limited to plasmids pcDNA3, pHCMV/Zeo, pCR3.1, pEF I/His, pEMD/GS, pRc/HCMV2, pSV40/Zeo2, pTRACER-HCMV, pUB6/V5-His, pVAXI, and pZeoSV2 (available from Invitrogen, San Diego, Calif.), and plasmid pCI (available from Promega, Madison, Wis.).

The nucleic acid molecule of the present invention are contemplated to be made primarily of RNA, though in some embodiments, they may be RNA, nucleotide analogs, DNA, or any combination of DNA, RNA, nucleotide analogs, and PNAs.

Maximizing activity of nucleic acid molecules of the invention which consist of or comprise the sequence AAAAGCUGGGUUGAGAGGGCGA as the “active” miRNA, requires maximizing uptake of the active strand (the third part and in particular the sequence AAAAGCUGGGUUGAGAGGGCGA) and minimizing uptake of the complementary strand (first part) by the miRNA protein complex that regulates gene expression at the level of translation. The molecular designs that provide optimal miRNA activity involve modifications to the complementary strand. The first modification involves creating a complementary strand (preferably RNA) with a chemical group other than a phosphate or hydroxyl at its 5′ terminus. The presence of the 5′ modification frequently eliminates uptake of the complementary strand and subsequently favours uptake of the active strand by the miRNA protein complex. The 5′ modification can be any of a variety of molecules including NH2, NHCOCH3, biotin, and others. The second chemical modification strategy that significantly reduces uptake of the complementary strand by the miRNA pathway is incorporating nucleotides with sugar modifications in the first 2-6 nucleotides of the complementary strand. It should be noted that the sugar modifications consistent with the second design strategy can be coupled with 5′ terminal modifications consistent with the first design strategy to further enhance synthetic miRNA activities. The third synthetic miRNA design involves incorporating nucleotides in the 3′ end of the complementary strand that are not complementary to the active strand. Such modifications and modification strategies are well known, explained for example in WO 2006/137941 and specifically encompassed by the embodiments of the present invention.

While native phosphodiester backbone linkages in the nucleic acid molecules of the present invention are preferred, other backbone linkages may be incorporated, e.g. backbone linkages containing a phosphorus atom. Modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

While it is likewise preferred that the nucleic acid molecules of the present invention comprise naturally occurring bases (naturally occurring bases include, for example, adenine, guanine, cytosine, thymine, uracil, and inosine), these bases may be modified. Modification may be by the replacement or addition of one or more atoms or groups. Some examples of types of modifications that can comprise nucleotides that are modified with respect to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O- and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides.

The present invention thus relates to the nucleic acid molecules of the invention, comprising one or more modifications selected from the modifications set forth herein before.

In a further embodiment, the nucleic acid molecules of the present invention comprise at least one detectable label, such as for example a radioactive or fluorescent moiety, or mass label attached to the nucleotide.

In a further embodiment, the present invention relates to a host cell comprising the nucleic acid molecule and/or the vector of the invention. The term “host cell” includes inter alia a bacterium (probiotic bacteria being preferred), preferably a gram-negative bacterium, more preferably a bacterium belonging to the family enterobacteriacea, and even more preferred a member of the genus Escherichia. In another preferred embodiment of the present invention, said host cell is a probiotic bacterium. Probiotic bacteria are, according to the definition set forth by the WHO bacteria associated with beneficial effects for humans and animals. The term “probiotic” further includes live, non-pathogenic microorganisms (preferably bacteria) which can confer a health benefit on the host, at least a health benefit for the gastrointestinal tract. Useful probiotics host cells include but are not limited to Bacillus coagulans, Bifidobacterium animalis subsp. Lactis, Bifidobacterium breve, Bifidobacterium infantis, Bifidobacterium animalis, Bifidobacterium longum, Escherichia coli M-17, Escherichia coli Nissle 1917, Lactobacillus acidophilus, Lactobacillus casei, Lactobacillus paracasei, Lactobacillus fortis, Lactobacillus johnsonii, Lactococcus lactis, Lactobacillus plantarum, Lactobacillus Lactobacillus reuteri, Lactobacillus rhamnosus, Lactobacillus rhamnosus, Saccharomyces cerevisiae, especially boulardii, Lactobacillus rhamnosus, Streptococcus thermophilus, Lactobacillus helveticus, mixtures thereof, and/or other bacteria of the above-listed genera.

In a particularly preferred embodiment, said probiotic host cell is selected from E. coli Nissle 1917 or E. coli 8178 DSM21844 (disclosed in WO2010/034479). The Escherichia coli strain Nissle 1917 is one of the best-studied probiotic strains. It is commercially available from ARDEYPHARM GmbH, Herdecke, Germany, under the trademark ‘Mutaflor’. This particular E. coli strain was isolated in 1917 by Alfred Nissle based on its potential to protect from infectious gastroenteritis. The Nissle 1917 strain has been shown to combine efficient intestinal survival and colonization with the lack of virulence. This makes it a safe and effective candidate in the treatment of inter alia chronic inflammatory bowel diseases as well as diarrheal diseases in young children.

In a preferred embodiment, the host cell of the present invention is for use as a medicament, and in particular for use in the treatment and/or amelioration, or prevention of a disease which disease is characterized by a reduction or loss of the intestinal barrier function as mediated by the intestinal mucosa. It is envisaged that the host cell and the nucleic acid molecules and/or vectors of the present invention, may coexist in the pharmaceutical composition of the present invention.

In another embodiment, the present invention relates to a composition (preferably a pharmaceutical composition) comprising a nucleic acid molecule of the invention and a probiotic bacterium, wherein the probiotic bacterium does neither comprise the nucleic acid molecule nor the vector of the present invention intracellularily.

The pharmaceutical composition of the present invention comprises a nucleic acid molecule, and/or vector and/or host cell according to the invention as an active ingredient and may further include a pharmaceutically acceptable carrier. A “pharmaceutically acceptable carrier” refers to an ingredient in a pharmaceutical formulation, other than an active ingredient, which is nontoxic to a subject. A pharmaceutically acceptable carrier includes, but is not limited to, a buffer (preferably an artificial buffer), excipient, stabilizer, and/or preservative. In regard to the treatment of colitis ulcerosa, it is particularly preferred that the pharmaceutical composition of the present invention comprises a buffer. In addition, the pharmaceutical composition of the invention may include other medicinal or pharmaceutical agents, adjuvants, etc. Exemplary parenteral administration forms include solutions or suspensions of active compounds) in sterile aqueous solutions, for example, aqueous propylene glycol or dextrose solutions. Such dosage forms can be suitably buffered, if desired. Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof. Methods of preparing various pharmaceutical compositions with a specific amount of active compound are known, or will be apparent, to those skilled in this art.

For examples—see Remington's Pharmaceutical Sciences, Mack Publishing Company, Ester, Pa., 15.sup.th Edition (1975). It will be understood, however, that the compositions of the invention may further comprise other components.

The (pharmaceutical) composition may, for example, be in a form suitable for oral administration as a tablet, capsule, pill, powder, sustained release formulations, solution, suspension, for parenteral injection as a sterile solution, suspension or emulsion or for rectal administration as a suppository. Oral administration is preferred, and as regards the treatment of colitis ulcerosa, oral administration is particularly preferred. The pharmaceutical composition may be in unit dosage forms suitable for single administration of precise dosages.

It is also envisaged that the nucleic acid molecules and/or vectors of the invention be provided in free form or bound to (for example covalently) and/or encompassed by a solid carrier, such as liposomes, nanotransporters, composites, metal complexes, polymers or biopolymers such as hydroxyapatite, nanoparticles, microparticles or any other vehicle considered useful for the delivery of nucleic acid molecules (including the vectors of the invention). The solid carrier comprising the nucleic acid molecule and/or vector of the present invention is preferably for use as a medicament, and in particular for use in the treatment and/or amelioration, or prevention of a disease which disease is characterized by a reduction or loss of the intestinal barrier function as mediated by the intestinal mucosa. A variety of compounds have been developed that complex with nucleic acids, deliver them to surfaces of cells, and facilitate their uptake in and release from endosomes. Among these are: (1) a variety of lipids such as DOTAP (or other cationic lipid), DDAB, DHDEAB, and DOPE and (2) non-lipid-based polymers like polyethylenimine, polyamidoamine, and dendrimers of these and other polymers. In certain of these embodiments a combination of lipids is employed such as DOTAP and cholesterol or a cholesterol derivative (U.S. Pat. No. 6,770,291, which is hereby incorporated by reference). Several of these reagents have been shown to facilitate nucleic acid uptake in animals and all these compounds or compounds having a comparable mode of action (i.e. facilitate the uptake of nucleic acid molecules into cells, preferably into human cells) are encompassed by the embodiments of the present invention.

A variety of compounds have been attached to the ends of nucleic acid molecules to facilitate their uptake/transport across cell membranes. Short signal peptides found in the HIV TAT, HSV VP22, Drosphila antennapedia, and other proteins have been found to enable the rapid transfer of biomolecules across membranes (reviewed by Schwarze 2000). These signal peptides, referred to as Protein Transduction Domains (PTDs), have been attached to oligonucleotides to facilitate their delivery into cultured cells. Cholesterols have been conjugated to oligonucleotides to improve their uptake into cells in animals (MacKellar 1992). The terminal cholesterol groups apparently interact with receptors or lipids on the surfaces of cells and facilitate the internalization of the modified oligonucleotides. Likewise, poly-1-lysine has been conjugated to oligonucleotides to decrease the net negative charge and improve uptake into cells (Leonetti 1990). All these entities which facilitate the uptake of nucleic acid molecules/vectors are also within the scope of the present invention.

In one embodiment, the compositions and/or the nucleic acid molecules and/or vectors and/or host cells (preferably the probiotic host cells) of the invention are supplied along with an ingestible support material for human consumption. Exemplary ingestible support materials include a cereal based food product, rice cake, soy cake, food bar product, cold formed food bar. The compositions and/or the nucleic acid molecules and/or vectors and/or host cells (preferably the probiotic host cells) discussed herein may be provided, for example, as dietary supplements, food and beverage additives, food and beverage ingredients.

It is also envisaged that the food or beverage products described herein above are intended for healthy subjects, preferably mammals and more preferably humans. Thus, the present invention also relates to the nucleic acid molecules and/or vectors and/or host cells and/or food or beverage product described herein (either individually or in admixture) for the supply of healthy subjects, and/or for promoting or conserving gut health or the wellbeing of a subject, preferably a human subject.

In a further embodiment, the present invention relates to a method of production of a food or beverage product, comprising the step of formulating the nucleic acid molecule, vector, host-cell and/or composition of the invention (either individually or in admixture) into a food or beverage product.

The present invention is also characterized by the following items:
Item 1. A nucleic acid molecule of up to 150 nucleotides comprising consecutively from 5′ to 3′:
   (a) a first part whose sequence is between 50% and 100% complementary to the sequence AAAAGCUGGGUUGAGAGGGCGA;
   (b) a second part capable of forming a loop between the first and the third part; and
   (c) a third part comprising or consisting of the sequence AAAAGCUGGGUUGAGAGGGCGA;
   for use as a medicament.
Item 2. The use of item 1, wherein the second part of the nucleic acid molecule is a nucleic acid sequence which is about 3 to 30 nucleotides in length, four nucleotides in length being preferred.
Item 3. The use of item 1 or 2 wherein the nucleic acid molecule is up to 85 nucleotides in length.
Item 4. The use of any one of the preceding items, wherein the first part of the nucleic acid molecule is at least 80% complementary to the sequence AAAAGCUGGGUUGAGAGGGCGA.
Item 5. The use of any one of the preceding items, wherein said nucleic acid molecule is capable of forming a stem-loop (a double helix that ends in an unpaired loop).
Item 6. The use of any one of the preceding items wherein the nucleic acid molecule comprises or consists (of) the sequence GCUUCGCUCCCCUCCGCCUUCUCUUCCCGGUUCUUCCCGGAGUCGGGAAAAGCUGGGUU GAGAGGGCGAAAAAGGAUGAGGU (hsa-mir-320a).
Item 7. The use of any one of the preceding items, wherein said nucleic acid molecule is processable by a mammalian cell (preferably a human cell) to the mature miRNA AAAAGCUGGGUUGAGAGGGCGA.
Item 8. A nucleic acid molecule of up to 25 nucleotides comprising the sequence AAAAGCUGGGUUGAGAGGGCGA, for use as a medicament.
Item 9. The use of any of the preceding items, wherein said nucleic acid molecule is RNA.
Item 10. A composition comprising at least one mature miRNA selected from the group consisting of hsa-miR-320a, ptr-miR-320a, ppy-miR-320a, bta-miR-320, cfa-miR-320, mmu-miR-320, rno-miR-320, and mml-miR-320, and/or one or more mir-RNA precursor(s) thereof, for use as a medicament.
Item 11. The use of any one of items 1 to 10, wherein said nucleic acid molecule and/or mature miRNA comprises one or more modifications.
Item 12. A vector comprising a nucleic acid molecule and/or mature miRNA as defined in any one of items 1 to 11, for use as a medicament.
Item 13. The use of item 12, wherein said vector is an expression vector.
Item 14. A host cell comprising the nucleic acid molecule, mature miRNA and/or the vector as defined in any one of the preceding items, for use as a medicament.
Item 15. The use of item 14, wherein said host cell is a bacterium, preferably a gram-negative bacterium, more preferably a bacterium belonging to the family enterobacteriacea.
Item 16. The use of item 15, wherein said bacterium is a probiotic bacterium.
Item 17. The use of item 16, wherein said probiotic bacterium is E. coli Nissle 1917 or E. coli 8178 DSM21844.
Item 18. A composition comprising a nucleic acid molecule and/or mature miRNA as defined in any one of the preceding claims and a probiotic bacterium as defined in item 17.
Item 19. The composition of item 18, further comprising E. coli Nissle 1917 and/or E. coli 8178 or a fraction thereof.
Item 20. The composition of item 18 or 19 for use as a medicament.
Item 21. A microparticle which is coated with the nucleic acid molecule, mature miRNA and/or the vector as defined in any one of the preceding items, for use as a medicament.
Item 22. The medicament as defined in any one of the preceding items, for use in the treatment of inflammatory bowel disease (IBD).
Item 23. The use as defined in any one of the preceding items, for the treatment of inflammatory bowel disease (IBD).
Item 24. The IBD as defined in item 22 or 23, wherein said IBD is ulcerative colitis, Cohn's disease, collagenous colitis, lymphocytic colitis, ischemic colitis, diversion colitis, Behçet disease, or indeterminate colitis.
Item 25. The medicament and/or use as defined in any one of the preceding items, which is for oral administration.
Item 26. A food product comprising the nucleic acid molecule, mature miRNA, vector, host cell, and/or microparticle as defined in any one of the preceding items.
Item 27. Use of the nucleic acid molecule, mature miRNA, vector, host cell, and/or microparticle as defined in any one of the preceding items for promoting gut health or the wellness of a subject.
Item 28. The use of item 27, wherein said subject is a normal healthy subject, preferably a normal healthy human.

The figures show:

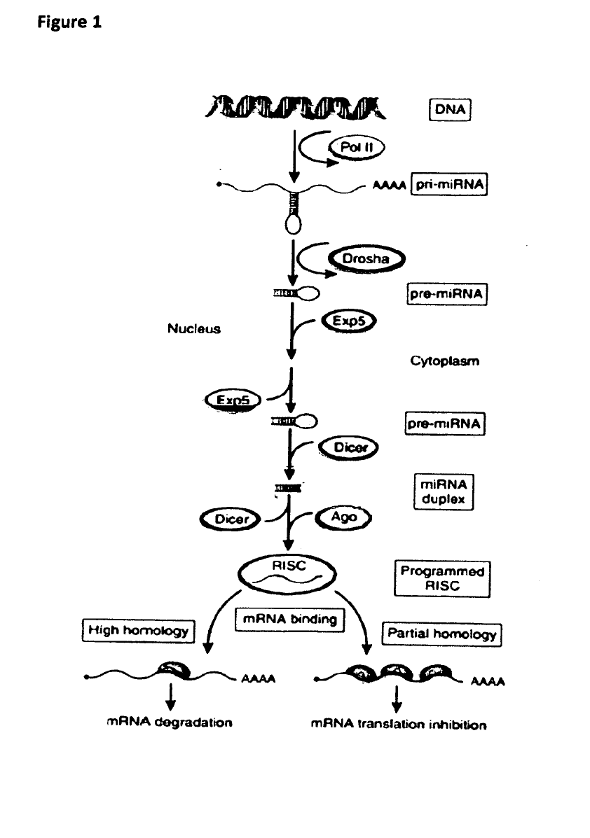

FIG. 1: Biogenesis and function of miRNAs

FIG. 2: Monitoring trans-epithelial electrical resistance (TER)

T84 cells were grown on Transwell filters for 8-10 days to 100% confluency. After reaching confluency, the filters were inserted into the appropriate wells of a recently developed cellZscope unit for real-time online TER-monitoring (NanoAnalytics, Munster, Germany) according to Karczewski et al. and Rempe et al. [45, 46]. The cellZscope monitors transepithelial impedance (ohmic resistance and capacitance) under physiological conditions without affecting the cellular barrier under investigation. The epithelial cells were infected with bacteria (MOI 100) in DMEM Ham's F12 plus FCS and incubated at 37° C./5% CO2. Changes in TER were monitored online for up to 40 h.

FIG. 3: principle of TER measurement

### EXAMPLES

The following examples illustrate the invention. These examples should not be construed as to limit the scope of this invention. The examples are included for purposes of illustration and the present invention is limited only by the claims.

### Example 1

### Co-Incubation of T84 Cells with EPEC Strain E2348/69 and EPEC+hsa mir-320a (See also FIG. 3)

T84 intestinal epithelial cells (ATCC CCL 248, passage 10-25) were grown in 5% CO2 at 37° C. The cells were cultured in collagen-coated flasks and tissue culture plates in DMEM Ham's F-12 (PAA, Cabe, Germany) complemented with 10% fetal calf serum (FCS) and antibiotics (100 μg/ml Penicillin/Streptomycin). To monitor trans-epithelial resistance (TER), T84 cells were cultured on Transwell filters (6.5 mm diameter, 0.4 μm pore size, Costar Corning, N.Y.).

T84 cells were incubated with E. coli and TER was measured of non-infected cells (control) and infected cells: T84 incubated without bacteria; T84 co-incubated with EPEC and T84 co-incubated with EPEC +has mir-320a. Online-monitoring was conducted using the CellZscope technology [NanoAnalytics, Munster, Germany].

The analysis of the TER serves as a fast and on-line measurable indicator for barrier-relevant alterations. With the parallel detection of ohmic and inductive resistance of the monolayer this system provides a reliable read-out of better quality than the conventionally employed measurement methods (Rempe et al., 2011).

The trans-epithelial electrical resistance (TER) and the capacitance (Ccl) of the monolayer will be detected by monitoring the frequency-dependent impedance (Z) (depicted here by an equivalent electronic circuit nanoAnalytics GmbH, Munster).

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, detailed Description, and Examples is hereby incorporated herein by reference.

1. A pharmaceutical composition comprising: a nucleic acid molecule of up to 150 nucleotides comprising consecutively from 5′ to 3′:
(a) a first part whose sequence is between 50% and 100% complementary to the sequence AAAAGCUGGGUUGAGAGGGCGA;
(b) a second part capable of forming a loop between the first and the third part; and
(c) a third part comprising or consisting of the sequence AAAAGCUGGGUUGAGAGGGCGA; and
a pharmaceutically acceptable carrier.

2. The pharmaceutical composition of claim 1, wherein said first part comprises at least 4 consecutive nucleotides of the sequence GCCUUCUCUUCCCGGUUCUUCCCG (from 5′to 3′).

3. A pharmaceutical composition of up to 25 nucleotides comprising the sequence AAAAGCUGGGUUGAGAGGGCGA; and a pharmaceutically acceptable carrier.

4. A pharmaceutical composition comprising: a host cell comprising
(i) a nucleic acid molecule of up to 150 nucleotides comprising consecutively from 5′ to 3′:
(a) a first part whose sequence is between 50% and 100% complementary to the sequence AAAAGCUGGGUUGAGAGGGCGA;
(b) a second part capable of forming a loop between the first and the third part; and
(c) a third part comprising or consisting of the sequence AAAAGCUGGGUUGAGAGGGCGA, or
(ii) a nucleic acid molecule of up to 25 nucleotides comprising the sequence AAAAGCUGGGUUGAGAGGGCGA; and
a pharmaceutically acceptable carrier.

5. The pharmaceutical composition of claim 4, wherein said host cell is a probiotic bacterium.

6. The pharmaceutical composition of claim 5, wherein said probiotic bacterium is E. coli Nissle 1917 or E. coli 8178 DSM21844.

7. The pharmaceutical composition according to claim 1, wherein the nucleic acid molecule is coated on a microparticle.

8. A method for the treatment of inflammatory bowel disease (IBD) comprising administering a therapeutically effective amount of the pharmaceutical composition according to claim 1.

9. The method according to claim 8, wherein said IBD is ulcerative colitis, Cohn's disease, collagenous colitis, lymphocytic colitis, ischemic colitis, diversion colitis, Behçet disease, or indeterminate colitis.

10. The pharmaceutical composition according to claim 1, wherein the composition is configured for oral administration.

11. The pharmaceutical composition according to claim 1, wherein the composition is configured as a food product.

12. A method for promoting or conserving gut health of a subject, wherein said subject is a normal healthy subject, preferably a normal healthy human, comprising administering a therapeutically effective amount of the pharmaceutical composition according to claim 1.

13. The pharmaceutical composition according to claim 3, wherein the nucleic acid molecule is coated on a microparticle.

14. A method for the treatment of inflammatory bowel disease (IBD) comprising administering a therapeutically effective amount of the pharmaceutical composition according to claim 3.

15. The method according to claim 8, wherein said IBD is ulcerative colitis, Cohn's disease, collagenous colitis, lymphocytic colitis, ischemic colitis, diversion colitis, Behçet disease, or indeterminate colitis.

16. The pharmaceutical composition according to claim 3, wherein the composition is configured for oral administration, and wherein the composition is optionally configured as a food product.

17. A method for promoting or conserving gut health of a subject, wherein said subject is a normal healthy subject, preferably a normal healthy human, comprising administering a therapeutically effective amount of the pharmaceutical composition according to claim 3.

18. A method for the treatment of inflammatory bowel disease (IBD) comprising administering a therapeutically effective amount of the pharmaceutical composition according to claim 4.

19. A method for promoting or conserving gut health of a subject, wherein said subject is a normal healthy subject, preferably a normal healthy human, comprising administering a therapeutically effective amount of the pharmaceutical composition according to claim 4.

20. The pharmaceutical composition according to claim 4, wherein the composition is configured for oral administration, and wherein the composition is optionally configured as a food product.
